(19) United States
(12) Patent Application Publication
(10) Pub. No.: US 2022/0110858 A1
(43) Pub. Date: Apr. 14, 2022

(54) AFFECTION GOLD HAIR CARE COMPOSITION AND METHOD

(71) Applicant: Ramona Nelshawn Lester, Mount Vernon, NY (US)

(72) Inventor: Ramona Nelshawn Lester, Mount Vernon, NY (US)

(21) Appl. No.: 17/450,639

(22) Filed: Oct. 12, 2021

Related U.S. Application Data
(60) Provisional application No. 63/090,377, filed on Oct. 12, 2020.

(51) Int. Cl. A61K 8/98; A61K 8/9789; A61K 8/49; A61Q 7/00

(52) U.S. Cl. CPC A61K 8/982; A61K 8/9789; A61K 2800/87; A61Q 7/00; A61K 8/4953

(57) ABSTRACT

The present invention provides a hair care composition comprising 2.5 fl. oz. 99% hemp oil; 2 fl. oz. 2% minoxidil; 2.5 fl. oz. 99% tea tree oil; 2.5 fl. oz. 99% peppermint oil; and 2 fl. oz. placenta. A hair care system may comprise a spray bottle; and a predetermined quantity of the hair care composition. A method for treating a user's hair may include the following steps: wash and dry a user's hair; apply a predetermined quantity of the hair care composition to the user's hair; massage the hair care composition thoroughly into the user's hair and leave the hair care composition in the user's hair.

### CROSS-REFERENCE TO RELATED APPLICATION

This application claims the benefit of priority of U.S. Provisional Application No. 63/090,377, filed Oct. 12, 2020, the contents of which are herein incorporated by reference.

### BACKGROUND OF THE INVENTION

The present invention relates to hair treatment, and more particularly, to a hair care composition and method.

Hair loss is a problem around the world, which leaves people feeling unhappy with their outer beauty.

Present options are few. For example, the cap light requires you to wear a cap. The cap light is expensive.

Other compositions are not attractive to be worn outdoors. Other compositions leave the hair dry, brittle, or hard, and may cause the user's hair to become dry and brittle.

Other liquid products are not suitable for all hair types.

A need exists for a solution which may be easy to use, versatile and capable of use on all hair types, cost-effective and economical. Further, a need exists for such a solution to be effective in promoting hair regrowth.

### SUMMARY OF THE INVENTION

Advantageously, in one aspect, the present invention provides a solution, which comprises a hair care composition and method. The present invention is easy to use, versatile, and capable of use on all hair types. The present invention is cost-effective and economical. Further, the composition is effective in promoting hair regrowth.

These and other features, aspects and advantages of the present invention will become better understood with reference to the following drawings, description and claims.

### BRIEF DESCRIPTION OF THE DRAWINGS

The preferred embodiment of the claimed subject matter will hereinafter be described in conjunction with the appended drawings provided to illustrate and not to limit the scope of the claimed subject matter, where like designations denote like elements, and in which:

FIG. 1 is a schematic view of the invention; and

FIG. 2 is a flow chart of the use of the invention.

It is to be understood that like reference numerals refer to like parts throughout the several views of the drawings.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

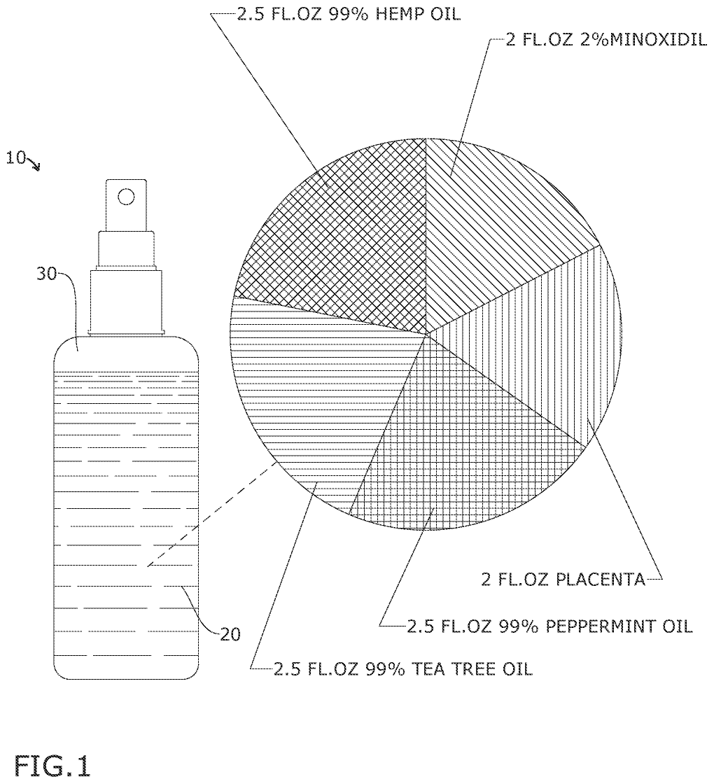

As used herein, “exemplary” or “illustrative” means “serving as an example, instance, or illustration.” Any implementation described herein as “exemplary” or “illustrative” is not necessarily to be construed as preferred or advantageous over other implementations. All the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, “vertical”, “horizontal”, and derivatives thereof shall relate to the invention as oriented in FIG. 1. Furthermore, as there is no intention to be bound by any express or implied theory presented in the preceding technical field, background, brief summary or the following detailed description, it is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Broadly, one embodiment of the present invention provides a hair care composition and a method for its use.

Advantageously, the present invention provides a solution, which comprises a hair care composition and method for its use. The present invention is easy to use and versatile. The present invention may be used and styled with the user's everyday hairdo. The present invention is capable of use on all hair types.

The present invention is cost-effective and economical. Further, the composition is effective in promoting hair regrowth. The present invention includes minoxidil which is sure to grow or regrow hair.

In an exemplary embodiment the composition may include one or more of the following components and combinations thereof.
Minoxidil
Hemp oil
Tea tree oil
Peppermint oil
Placenta

In an exemplary embodiment the composition may be made by a process including one or more of the following steps and combinations thereof. Provide the following components:
1. Minoxidil 2%=Hair Growth
2. 99% Hemp Oil=Hair Strength
3. 99% Tea Tree Oil=Prevent build of chemicals
4. 99% Peppermint Oil=Open the pores for circulation allowing growth to start
5. Placenta protein=Repairs damaged hair.
The components may be provided in the following amounts:
2.5 fl. oz. 99% hemp oil
2 fl. oz. 2% minoxidil
2.5 fl. oz. 99% tea tree oil
2.5 fl. oz. 99% peppermint oil
2 fl. oz. placenta

FIG. 1 shows a schematic view of the invention. As seen at FIG. 1, the invention, referred to generally at 10, may provide a hair care composition 20 which may be provided in a spray bottle 30.

Also seen at FIG. 1, the composition 20 may include one or more of the following components and combinations thereof:
2.5 fl. oz. 99% hemp oil
2 fl. oz. 2% minoxidil
2.5 fl. oz. 99% tea tree oil
2.5 fl. oz. 99% peppermint oil
2 fl. oz. placenta

The components are combined in these amounts to provide a quantity of 11.5 fluid ounces of the hair care composition. It can be seen that greater volumes of the hair care composition may be provided in the same ratios or proportions.

In an exemplary embodiment of the present invention, compositions of the present invention may be used in a process including one or more of the following steps and combinations thereof.
1. Wash hair
2. Towel dry
3. Shake Well
4. Apply one or two sprays in hand
5. Massage thoroughly
6. Leave in hair, do not rinse,
7. Style hair
8. Continued use is recommended to increase hair regrowth and keep the hair regrowth, or hair loss may occur again.

In an exemplary embodiment these method steps may interrelate in the following manner. Steps 1 and 2 are important cleanse the scalp prior to use to improve effectiveness of the composition. Step 3 is important to ensure the components are mixed for use. Steps 4-6 are important for the managed care of the hair and the growth process. Step 7 is optional. Step 8 is the continued application to maintain growth.

In an exemplary embodiment the present invention works in the following manner. A user of the composition would follow all the steps listed in order and continue to use the product to maintain the benefits achieved. A user may experience noticeable hair growth, improved hair appearance, and feel testing. Hair regrowth time may differ for each user. If a user does not see regrowth in 6 months the user should consult a doctor.

Hair regrowth time may be different for each user. If a user does not see regrowth in 6 months, the user should consult a doctor. If the user is satisfied after 6 months no other tests are needed and the user may continue use to maintain the hair regrowth. Testing may also be provided for a user with no hair loss problems which will still cause the hair to grow or regrow if damaged.

In an exemplary embodiment of the present invention, compositions of the present invention may be used in a process including one or more of the following steps and combinations thereof.
Wash hair
Dry hair
Apply a predetermined quantity of the composition in hand or to problem area
Massage the quantity of the composition thoroughly into hair
Leave in hair, do not rinse
Style hair
Continue use of composition as recommended to increase hair regrowth and keep hair regrowth

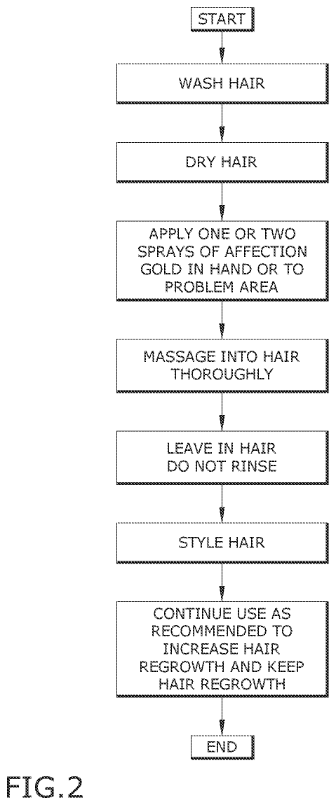

FIG. 2 is a flow chart of the use of the invention. The present invention may be used in a process including one or more of the following steps and combinations thereof.
Provide a hair care composition as described herein
Wash hair
Dry hair
Apply one or two sprays of the hair care composition in the hand or to the problem area
Massage the composition thoroughly into the hair
Leave the composition in the hair
Do not rinse
Style hair
Continue use of the composition as recommended to increase hair growth and keep hair regrowth.

In summary, the present invention provides a composition and method for hair care. The composition may comprise the following components: hemp oil; minoxidil; tea tree oil; peppermint oil; and placenta.

The hair care composition may comprise 2.5 fl. oz. 99% hemp oil; 2 fl. oz. 2% minoxidil; 2.5 fl. oz. 99% tea tree oil; 2.5 fl. oz. 99% peppermint oil; and 2 fl. oz. placenta.

The present invention provides a method for treating a user's hair, the method comprising the following steps: provide a hair care composition as described herein; wash a user's hair; dry the user's hair; apply a predetermined quantity of the hair care composition to the user's hair; massage the hair care composition thoroughly into the user's hair; leave the hair care composition in the user's hair; and do not rinse the hair care composition from the user's hair. The method may include the optional step of styling the user's hair. The method may comprise the additional step of continuing daily use of the composition to increase hair growth and maintain hair regrowth.

The present invention provides a hair care system comprising a spray bottle; and a predetermined quantity of a hair care composition described herein.

It should be understood, of course, that the foregoing relates to exemplary embodiments of the invention and that modifications, variations and changes in detail may be made without departing from the spirit and scope of the invention as set forth in the following claims.

What is claimed is:

1. A hair care composition comprising the following components:
a. hemp oil;
b. minoxidil;
c. tea tree oil;
d. peppermint oil; and
e. placenta.

2. The hair care composition of claim 1 wherein the components are provided in the following quantities:
a. 2.5 fl. oz. 99% hemp oil;
b. 2 fl. oz. 2% minoxidil;
c. 2.5 fl. oz. 99% tea tree oil;
d. 2.5 fl. oz. 99% peppermint oil; and
e. 2 fl. oz. placenta.

3. A method for treating a user's hair, the method comprising the following steps:
a. provide a hair care composition as in claim 2;
b. wash a user's hair;
c. dry the user's hair;
d. apply a predetermined quantity of the hair care composition to the user's hair;
e. massage the hair care composition thoroughly into the user's hair;
f. leave the hair care composition in the user's hair; and
g. do not rinse the hair care composition from the user's hair.

4. The method of claim 3 comprising the following additional step:
style the user's hair.

5. The method of claim 3 comprising the following additional step:
continue daily use of the composition to increase hair growth and maintain hair regrowth.

6. A hair care system comprising:
a. a spray bottle; and
b. a predetermined quantity of a hair care composition as in claim 2.
